(19) United States
(12) Patent Application Publication
(10) Pub. No.: US 2019/0186070 A1
(43) Pub. Date: Jun. 20, 2019

(54) HEATING APPARATUS FOR DEVELOPING LATENT IMPACT MARK

(71) Applicant: Republic of Korea (National Forensic Service Director Ministry of Public Administration and, Seoul (KR)

(72) Inventors: Dong A Lim, Yuseong-gu (KR); Kyeong Mi Kim, Namyangju-si (KR); Hyo Seon Han, Wonju-si (KR); Young ll Seo, Wonju-si (KR); Sang Yoon Lee, Siheung-si (KR); Eun Ah Joo, Yongin-si (KR); Jin Pyo Kim, Yuseong-gu (KR); Jae Mo Goh, Wonju-si (KR); Nam Kyu Park, Bucheon-si (KR)

(21) Appl. No.: 16/156,815

(22) Filed: Oct. 10, 2018

(30) Foreign Application Priority Data
Dec. 14, 2017 (KR) 10-2017-0172506

(51) Int. Cl. D06F 71/34; D06F 71/02

(52) U.S. Cl. CPC D06F 71/34; G01N 33/68; D06F 71/026

(57) ABSTRACT

Provided herein is a heating apparatus for developing a latent impact mark. The heating apparatus includes: a sample treated with an amino acid reagent containing amino acids capable of reacting with a latent impact mark; a first heating plate having a plate shape, having a first steam hole in a lower surface thereof, the first steam hole allowing steam to be discharged therethrough, and capable of being heated to a predetermined temperature; a second heating plate having a plate shape, having a second steam hole in an upper surface thereof, the second steam hole allowing steam to be discharged therethrough, and capable of being heated to a predetermined temperature; and a connecting portion configured to connect the first heating plate to the second heating plate such that the first heating plate is rotatable with respect to the second heating plate.

### CROSS-REFERENCE TO RELATED APPLICATION

This application claims the benefit of Korean Patent Application No. 10-2017-0172506, filed on Dec. 14, 2017, in the Korean Intellectual Property Office, the disclosure of which is incorporated herein in its entirety by reference.

### BACKGROUND

### 1. Field

One or more embodiments relate to a heating apparatus for developing a latent impact mark, and more particularly, to a heating apparatus for developing a latent impact mark, which is capable of precisely and rapidly analyzing an impact mark by placing a sample treated with an amino acid reagent between a first heating plate and a second heating plate and heating the sample, and identifying whether or not the impact mark is developed, without wrinkles and in a short time period.

### 2. Description of the Related Art

When a victim is assaulted with a criminal tool having an impact such as that in an assault at a crime scene, latent impact marks appear on an inner surface of the victim's clothes. As such, latent impact marks that may appear on the inner surface of the victim's clothes may be developed using an amino acid reagent. Thus, various types of amino acid reagents may be applied and then wet heat treatment using a heat iron may be performed to identify the presence or absence of development of a tool type using visual and optical methods.

However, existing apparatuses for developing latent impact marks have the following problems. Wet heat treatment is required to develop latent impact marks on an inner surface of the victim's clothes, and existing research institutes have used general heat irons sold in the market. However, in the case of large-sized samples, such general heat irons perform inaccurate heat treatment on the samples when processed, resulting in reduced developability, and may apply wrinkles or the like to a sample.

In addition, to detect a latent impact mark, both upper and lower surfaces of a sample must be heat-treated, and existing general heat irons are unable to heat-treat both the upper and lower surfaces of a sample at the same time. Therefore, to develop a latent impact mark, heat treatment must be performed separately on the upper and lower surfaces of a sample by using an existing general heat iron, and thus, much time is required to develop a latent impact mark, and precise heat treatment is not able to be performed.

### SUMMARY

One or more embodiments include a heating apparatus for developing a latent impact mark, which is capable of accurately and rapidly analyzing an impact mark by placing a sample treated with an amino acid reagent between a first heating plate and a second heating plate and heating the sample, and identifying the presence or absence of development of the impact mark without wrinkles and in a short time period.

Additional aspects will be set forth in part in the description which follows and, in part, will be apparent from the description, or may be learned by practice of the presented embodiments.

According to one or more embodiments, a heating apparatus for developing a latent impact mark includes a sample treated with an amino acid reagent containing amino acids capable of reacting with a latent impact mark; a first heating plate having a plate shape, having a first steam hole in a lower surface thereof, the first steam hole allowing steam to be discharged therethrough, and capable of being heated to a predetermined temperature; a second heating plate having a plate shape, having a second steam hole in an upper surface thereof, the second stem hole allowing steam to be discharged therethrough, and capable of being heated to a predetermined temperature; and a connecting portion configured to connect the first heating plate to the second heating plate such that the first heating plate is rotatable with respect to the second heating plate, wherein the sample is inserted between the first heating plate and the second heating plate to be heated simultaneously by the first heating plate and the second heating plate.

The heating apparatus may further include a temperature controller provided on the first heating plate or the second heating plate and capable of controlling a heating temperature of each of the first heating plate and the second heating plate; and a steam controller provided on the first heating plate or the second heating plate and capable of controlling an amount of steam discharged via the first steam hole of the first heating plate and the second steam hole of the second heating plate.

The heating apparatus may further include a locking device configured to perform a fixing operation such that the first heating plate is not rotatable with respect to the second heating plate, and when the locking device performs a fixing operation such that the first heating plate is not rotatable with respect to the second heating plate, the first heating plate and the second heating plate start heating up.

The heating apparatus may further include a first heating steam button capable of controlling operations of heating the first heating plate and discharging steam via the first steam hole, and a second heating steam button capable of controlling operations of heating the second heating plate and discharging steam via the second steam hole, and a size of each of the first heating plate and the second heating plate may be larger than that of the sample.

In the heating apparatus, a protrusion may be provided on the upper surface of the second heating plate, the protrusion protruding from the upper surface of the second heating plate and capable of preventing movement of the sample, and a protrusion groove may be provided in the lower surface of the first heating plate, the protrusion groove having a shape corresponding to that of the protrusion such that the protrusion is insertable into the protrusion groove.

The first steam hole and the second steam hole may have different sizes, and the first heating plate and the second heating plate may be covered with synthetic fiber.

### BRIEF DESCRIPTION OF THE DRAWINGS

These and/or other aspects will become apparent and more readily appreciated from the following description of the embodiments, taken in conjunction with the accompanying drawings in which:

FIG. 1 a schematic view of a heating apparatus for developing a latent impact mark, according to an embodiment;

FIG. 2 is a side view of a heating apparatus for developing a latent impact mark, according to an embodiment;

FIG. 3 is a front view of a heating apparatus for developing a latent impact mark, according to an embodiment;

FIG. 4A is a view illustrating a power supply unit, a temperature controller, a steam controller, and a locking device, according to an embodiment;

FIG. 4B is a view illustrating a first heating steam button and a second heating steam button, according to an embodiment;

FIG. 5 is a view illustrating a first heating plate and a second heating plate that heat up and discharge steam through an operation of a locking device, according to an embodiment;

FIG. 6 is a view illustrating a state in which a sample is placed in a heating apparatus for developing a latent impact mark, according to an embodiment; and

FIG. 7 is a view illustrating protrusions provided in a second heating plate and protrusion grooves provided in a first heating plate, according to an embodiment.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. Expressions such as “at least one of,” when preceding a list of elements, modify the entire list of elements and do not modify the individual elements of the list.

The present disclosure relates to a heating apparatus for developing a latent impact mark, which is capable of precisely and rapidly analyzing an impact mark by placing a sample treated with an amino acid reagent between a first heating plate and a second heating plate and heating the sample and identifying the presence or absence of development of the impact mark without wrinkles within a short time period. The present disclosure relates to a large-scale instantaneously heating apparatus for developing an impact mark, which is devised to more precisely and rapidly analyze an impact mark, and the apparatus may develop an impact mark in wet heat treatment of a sample without wrinkles within a short time period. Hereinafter, example embodiments of the present disclosure will be described in detail with reference to the accompanying drawings.

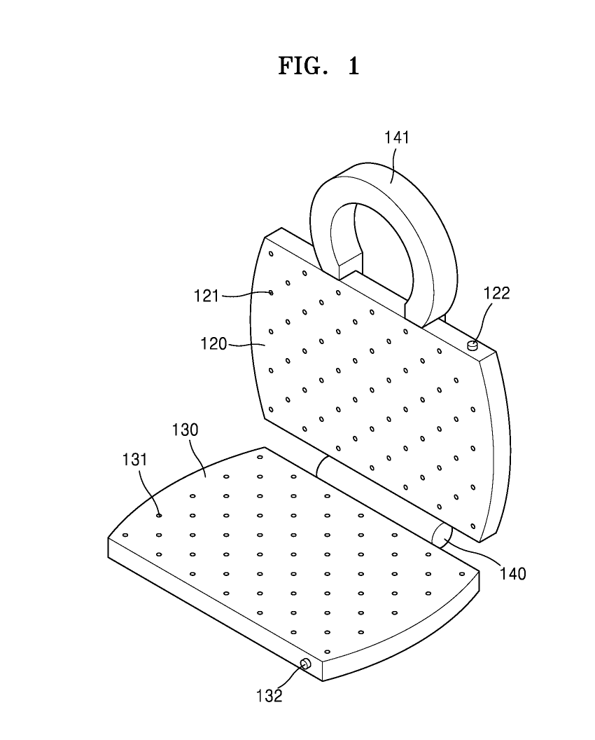

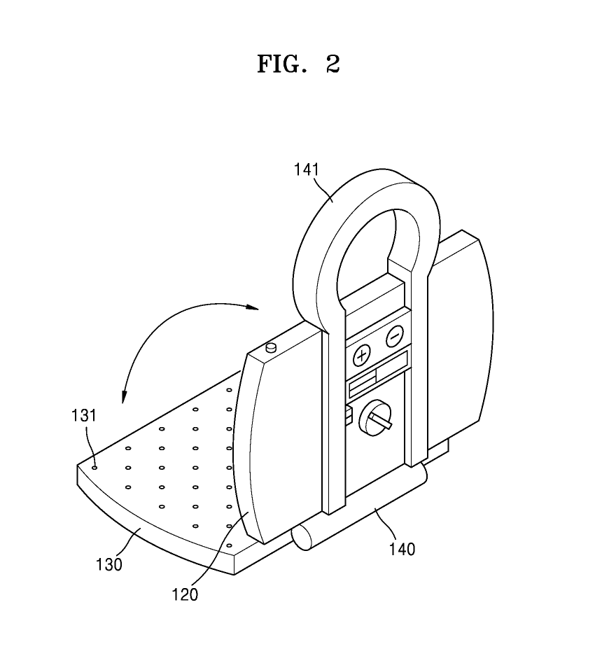

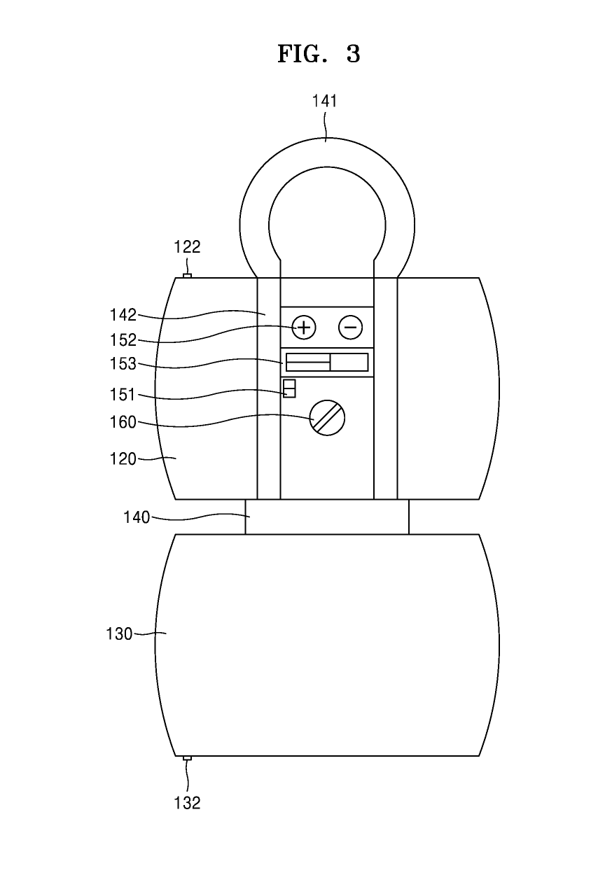

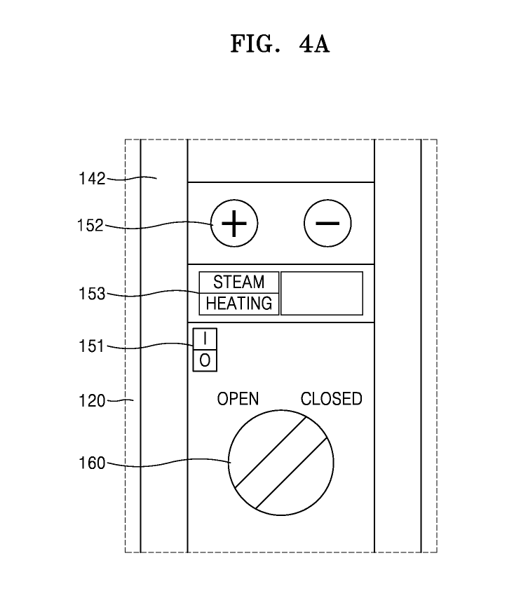

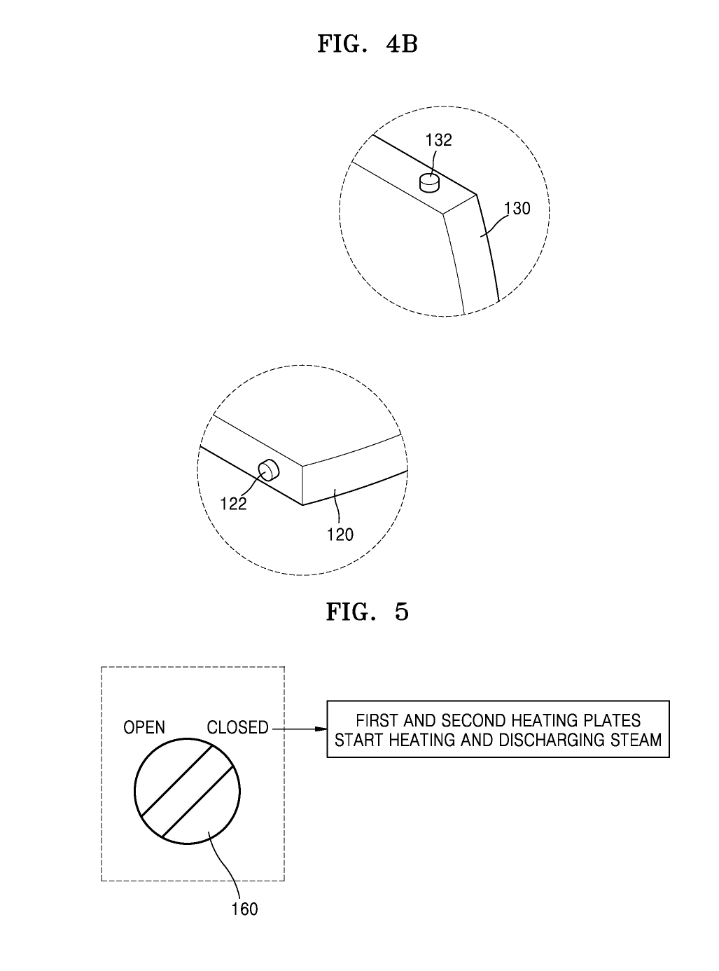

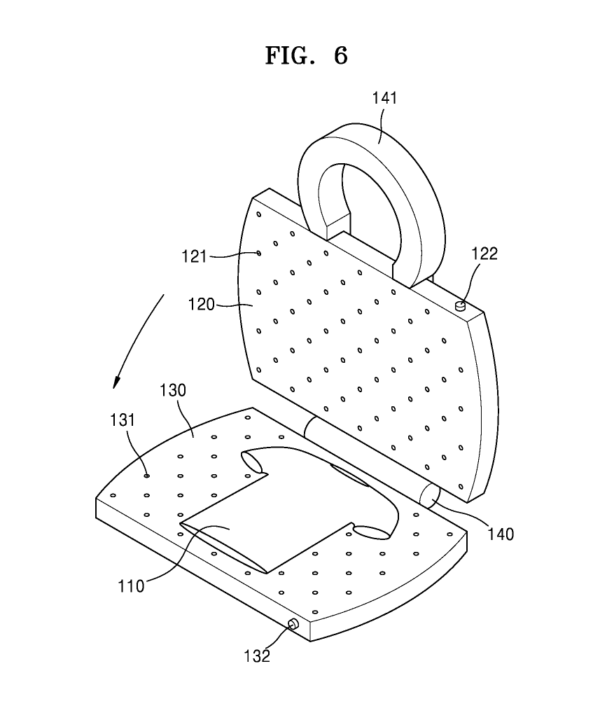

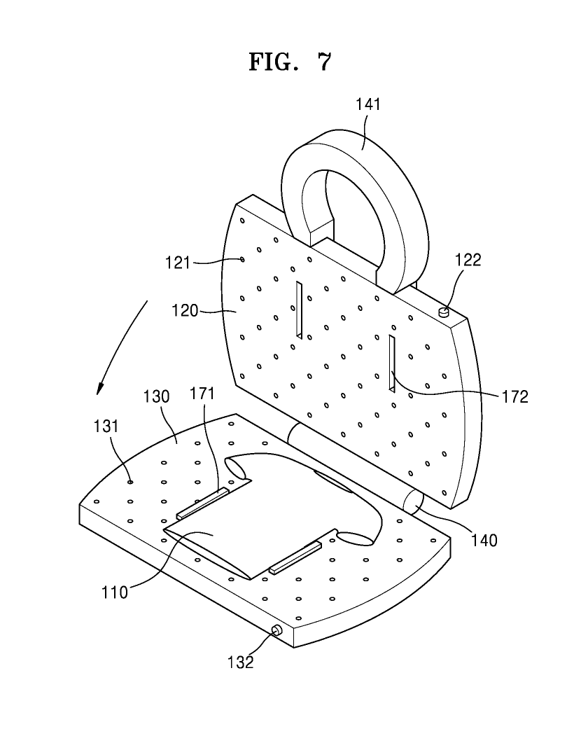

FIG. 1 a schematic view of a heating apparatus for developing a latent impact mark, according to an embodiment. FIG. 2 is a side view of a heating apparatus for developing a latent impact mark, according to an embodiment. FIG. 3 is a front view of a heating apparatus for developing a latent impact mark, according to an embodiment. FIG. 4A is a view illustrating a power supply unit, a temperature controller, a steam controller, and a locking device, according to an embodiment. FIG. 4B is a view illustrating first and second heating steam buttons, according to an embodiment. FIG. 5 is a view illustrating first and second heating plates that heat up and discharge steam through an operation of a locking device, according to an embodiment. FIG. 6 is a view illustrating a state in which a sample is located in a heating apparatus for developing a latent impact mark, according to an embodiment. FIG. 7 is a view illustrating protrusions provided in a second heating plate and protrusion grooves provided in a first heating plate, according to an embodiment.

According to an embodiment, the heating apparatus for developing a latent impact mark may include a sample 110, a first heating plate 120, a second heating plate 130, and a connecting portion 140.

The sample 110 is treated with an amino acid reagent containing amino acids capable of reacting with a latent impact mark. When a victim is assaulted by a criminal tool that applies an impact such as an assault at a crime scene, a skin secretion is transferred to a piece of clothing or the like and as a result, a latent impact mark remains thereon. Such a latent impact mark may be developed by reacting with amino acids and then undergoing wet heat treatment.

The sample 110 may be a piece of clothing or the like on which such a latent impact mark may remain. However, the sample 110 is not limited thereto, and may include various samples as long as they allow a skin secretion to be transferred thereto by an impact such as an assault while being in contact with the skin so that a latent impact mark remains thereon. The sample 110 is treated with an amino acid reagent containing amino acids capable of reacting with a latent impact mark, and is simultaneously heated by the first heating plate 120 and the second heating plate 130, which will be described below, so that a latent impact mark is developed on the sample 110.

Referring to FIG. 1, the first heating plate 120 has a plate shape, and is provided, in a lower surface thereof, first steam holes 121 capable of discharging steam therethrough. The first heating plate 120 is configured to provide heat and steam for the sample 110, and the lower surface of the first heating plate 120 comes into contact with an upper surface of the sample 110.

Since the lower surface of the first heating plate 120 comes into contact with the upper surface of the sample 110, heat and steam may be discharged from the lower surface of the first heating plate 120. Thus, the first steam holes 121, through which steam may be discharged, are provided in the lower surface of the first heating plate 120. Steam is discharged via the first steam holes 121, and the discharged steam is supplied to the upper surface of the sample 110.

The first heating plate 120 has a heat source such as a heating wire therein to allow the first heating plate 120 to be heated up to a predetermined temperature. Here, since the sample 110 comes into contact with the lower surface of the first heating plate 120, heat supplied by the first heating plate 120 may be supplied only to the lower surface of the first heating plate 120, not to an upper surface thereof. For this, a heat insulating material or the like may be provided on a portion of the inside of the first heating plate 120, facing the upper surface thereof.

Referring to FIG. 1, the second heating plate 130 has a plate shape, and is provided, in an upper surface thereof, with second steam holes 131 through which steam may be discharged. The second heating plate 130 is configured to provide heat and steam for the sample 110, and the upper surface of the second heating plate comes into contact with a lower surface of the sample 110.

Since the upper surface of the second heating plate 130 comes into contact with the upper surface of the sample 110, heat and steam may be discharged from a lower surface of the second heating plate 130. Thus, the second steam holes 131 through which steam may be discharged are provided in the lower surface of the second heating plate 130. Steam is discharged via the second steam holes 131, and the discharged steam is supplied to the lower surface of the sample 110.

The second heating plate 130 has a heat source such as a heating wire therein to allow the second heating plate 130 to be heated up to a predetermined temperature. Here, since the sample 110 comes into contact with the upper surface of the second heating plate 130, heat to be supplied to the second heating plate 130 may be supplied only to the upper surface of the second heating plate 130, not to the lower surface thereof. For this, a heat insulating material or the like may be provided on a portion of the inside of the second heating plate 130, facing the lower surface thereof.

That is, the sample 110 is inserted between the first heating plate 120 and the second heating plate 130, and is heated simultaneously by the first heating plate 120 and the second heating plate 130. The upper surface of the sample 110 is heated while being in contact with the lower surface of the first heating plate, the lower surface of the sample 110 is heated while being in contact with the upper surface of the second heating plate 130, and at the same time, wet heat treatment of the sample 110 is enabled by receiving steam via the first steam holes 121 and the second steam holes 131.

As such, to perform wet heat treatment on the sample 110 inserted between the first heating plate 120 and the second heating plate 130, the first heating plate 120 may be rotatably moved with respect to the second heating plate 130. Only when the first heating plate 120 is rotatably moved with respect to the second heating plate 130, the first heating plate 120 may be lifted to insert the sample 110 between the first heating plate 120 and the second heating plate 130, and after the sample 110 is inserted, the sample 110 is covered by the first heating plate 120 and may be heated simultaneously by the first heating plate 120 and the second heating plate 130.

The connecting portion 140 is configured to connect the first heating plate 120 to the second heating plate 130 so that the first heating plate 120 is rotatable with respect to the second heating plate 130. In particular, referring to FIG. 2, the connecting portion 140 is coupled to an end of a side surface of the first heating plate 120, and is simultaneously coupled to an end of a side surface of the second heating plate 130. In this regard, the connecting portion 140 may be formed as a hinge such that the first heating plate 120 is rotatable with respect to the second heating plate 130.

When the connecting portion 140 is formed as a hinge, the first heating plate 120 is rotatable with respect to the second heating plate 130, and the connecting portion 140 enables the first heating plate 120 to be opened or closed with respect to the second heating plate 130. When the first heating plate 120 is opened with respect to the second heating plate 130, the sample 110 is placed on the upper surface of the second heating plate 130, and when the first heating plate 120 is closed with respect to the second heating plate 130, the sample 110 may be supplied with heat and steam simultaneously from the first heating plate 120 and the second heating plate 130.

However, the connecting portion 140 is not limited to the hinge, and may include various devices as long as they allow the first heating plate 120 to be rotatable with respect to the second heating plate 130.

To apply a moving force to the first heating plate 120 when rotatably moving the first heating plate 120 with respect to the second heating plate 130, a handle 141 may be provided in the first heating plate 120. A user may apply a force to the first heating plate 120 via the handle 141, and thus the first heating plate 120 may be moved rotatably with respect to the second heating plate 130. That is, the first heating plate 120 may be opened or closed with respect to the second heating plate 130 via the handle 141.

The handle 141 may have various shapes as long as they allow a force to be applied to the first heating plate 120, and may be coupled to various positions of the first heating plate 120 as long as they allow a force to be applied to the first heating plate 120.

The size of each of the first heating plate 120 and the second heating plate 130, which have a plate shape, may be larger than that of the sample 110. Existing heating apparatuses cannot simultaneously heat the upper and lower surfaces of the sample 110, and exhibit reduced developability due to imprecise heat treatment in processing when the size of the sample 110 is large. According to an embodiment, to prevent this, the size of each of the first and second heating plates 120 and 130 may be larger than that of the sample 110.

In particular, the first heating plate 120 may be larger than the sample 110 such that the first heating plate 120 covers the upper surface of the sample 110, and the second heating plate 130 may be larger than the sample 110 such that the second heating plate 130 covers the lower surface of the sample 110 (see FIG. 6). Accordingly, the upper and lower surfaces of the sample 110 may be simultaneously heated, and wet heat treatment may be precisely performed by applying heat to the entire surface of the sample 110.

In addition, the first heating plate 120 and the second heating plate 130 may be covered with synthetic fiber (fabric). In particular, the lower surface of the first heating plate 120, contacting the sample 110 and the upper surface of the second heating plate 130, contacting the sample 110 may be covered with synthetic fiber (fabric). The first heating plate 120 and the second heating plate 130 use a material with rigidity such as steel to perform a heating operation, and such a material may damage the sample 110. Thus, the first heating plate 120 and the second heating plate 130 are covered with synthetic fiber, thereby preventing the sample 110 from being damaged by the first heating plate 120 and the second heating plate 130.

According to an embodiment, the heating apparatus for developing a latent impact mark may further include a power supply unit 151, a temperature controller 152, a steam controller 153, a locking device 160, a first heating steam button 122, and a second heating steam button 132.

The power supply unit 151, the temperature controller 152, the steam controller 153, and the locking device 160 may be arranged on at least one of the first heating plate 120 and the second heating plate 130, and may be arranged on the upper surface of the first heating plate 120. The lower surface of the second heating plate 130 is in contact with the bottom and the upper surface of the second heating plate 130 is a position that enables heat and steam to be supplied to the sample 110, and thus when the power supply unit 151, the temperature controller 152, the steam controller 153, and the locking device 160 are arranged on the second heating plate 130, a user may feel inconvenient during operation. Thus, for the convenience of a user, the power supply unit 151, the temperature controller 152, the steam controller 153, and the locking device 160 may be arranged on the first heating plate 120.

FIG. 3 is a front view of a heating apparatus for developing a latent impact mark, according to an embodiment. FIG. 3 illustrates that the power supply unit 151, the temperature controller 152, the steam controller 153, and the locking device 160 are arranged on the upper surface of the first heating plate 120, and hereinafter, the case in which the power supply unit 151, the temperature controller 152, the steam controller 153, and the locking device 160 are arranged on the first heating plate 120 will be described. However, the present disclosure is not limited to the above case, and the power supply unit 151, the temperature controller 152, the steam controller 153, and the locking device 160 may also be arranged on the second heating plate 130.

Referring to FIGS. 3 and 4A, the power supply unit 151 is configured to turn on and off a power supply of the heating apparatus for developing a latent impact mark. The power supply unit 151 may include an In (I) button and an Out (0) button, and the power supply of the heating apparatus for developing a latent impact mark is turned on when the I button is pressed, and the power supply of the heating apparatus for developing a latent impact mark is turned off when the 0 button is pressed. However, the power supply unit 151 is not limited to the above example, and may include various devices as long as they enable the power supply of the heating apparatus for developing a latent impact mark to be turned on and off.

The temperature controller 152 is configured to adjust a heating temperature of each of the first heating plate 120 and the second heating plate 130. The first heating plate 120 and the second heating plate 130 may be heated to a predetermined temperature, and the predetermined temperature may vary according to an amino acid reagent used in the sample 110. The temperature controller 152 is configured to adjust a predetermined temperature that may vary according to an amino acid reagent used in the sample 110.

Referring to FIG. 4A, the temperature controller 152 may include a (+) button configured to raise temperature and a (−) button configured to decrease temperature. In addition, the temperature controller 152 may control the temperature of the first heating plate 120 and the temperature of the second heating plate 130 at the same time, or may be provided with individual buttons configured to control the temperatures of the first and second heating plates 120 and 130 such that the respective temperatures of the first heating plate 120 and the second heating plate 130 may be controlled thereby.

The steam controller 153 is configured to control the amount of steam discharged via the first steam holes 121 of the first heating plate 120 and the second steam holes 131 of the second heating plate 130. Steam may be discharged to the sample 110 via the first steam holes 121 of the first heating plate 120 and the second steam holes 131 of the second heating plate 130, and the amount of steam may vary according to an amino acid reagent used in the sample 110.

The steam controller 153 is configured to control the amount of steam that may vary according to an amino acid reagent used in the sample 110. Referring to FIG. 4A, the steam controller 153 may control, at the same time, the amount of steam discharged via the first steam holes 121 of the first heating plate 120 and the amount of steam discharged via the second steam holes 131 of the second heating plate 130, or may separately control the amount of steam discharged via the first steam holes 121 of the first heating plate 120 and the amount of steam discharged via the second steam holes 131 of the second heating plate 130.

Referring to FIG. 4B, the heating apparatus may further include the first heating steam button 122 configured to control operations of heating the first heating plate 120 and discharging steam via the first steam holes 121 and the second heating steam button 132 configured to control operations of heating the second heating plate 130 and discharging steam via the second steam holes 131.

When the heating temperature and steam amount of each of the first heating plate 120 and the second heating plate 130 are predetermined by the temperature controller 152 and the steam controller 153, the first heating steam button 122 and the second heating steam button 132 may respectively control the operations of the first heating plate 120 and the second heating plate 130.

When the first heating steam button 122 is pressed, heating and discharging steam of the first heating plate 120 may be performed, and when the second heating steam button 132 is pressed, heating and discharging steam of the second heating plate 130 may be performed. In this regard, the first heating plate 120 and the second heating plate 130 may be independently controlled through the first heating steam button 122 and the second heating steam button 132. When only the first heating steam button 122 is pressed and the second heating steam button 132 is not pressed, only the first heating plate 120 may heat up and discharge steam, and when only the second heating steam button 132 is pressed and the first heating steam button 122 is not pressed, only the second heating plate 130 may heat up and discharge steam.

The upper and lower surfaces of the sample 110 may be simultaneously heated and steam may be discharged thereto, and in some embodiments, only the upper surface or lower surface of the sample 110 needs to be heated. In this case, the first heating steam button 122 and the second heating steam button 132 may be used.

Referring to FIGS. 4A and 5, the locking device 160 performs a fixing operation such that the first heating plate 120 is not rotatable with respect to the second heating plate 130. In particular, the locking device 160 may perform a fixing operation by controlling an operation of the connecting portion 140 so that the first heating plate 120 is not rotatable with respect to the second heating plate 130. For example, when the connecting portion 140 is formed as a hinge, a fixing operation may be performed by connecting a device that does not allow rotation of the hinge to the locking device 160 so that the first heating plate 120 is not rotatable with respect to the second heating plate 130.

The locking device 160 may be in an “open” state or a “closed” state. Here, when the locking device 160 is in the “open” state, the first heating plate 120 may be in a state in which the first heating plate 120 is rotatable with respect to the second heating plate 130, and when the locking device 160 is in the “closed” state, the first heating plate 120 may be in a state in which the first heating plate 120 is not rotatable with respect to the second heating plate 130. In a case in which the sample 110 is inserted between the first heating plate 120 and the second heating plate 130, and heat and steam are supplied to the sample 110, if the first heating plate 120 is moved, there is a risk of imprecise heat treatment. As such, when heat and steam are supplied to the sample 110, the locking device 160 may fix movement of the first heating plate 120, thereby allowing precise heat treatment to be performed on the sample 110.

The locking device 160 must be operated at a point at which heat and steam are supplied to the sample 110 by the first heating plate 120 and the second heating plate 130, the locking device 160 may be operated in conjunction with a point at which the first heating plate 120 and the second heating plate 130 start heating up.

An embodiment of the present disclosure will now be described with reference to FIG. 5. When the locking device 160 performs a fixing operation such that the first heating plate 120 is not rotatable with respect to the second heating plate 130, the first heating plate 120 and the second heating plate 130 may be controlled such that the first and second heating plates 120 and 130 start heating up. That is, when the locking device 160 is operated in the “closed” state, movement of the first heating plate 120 is fixed, and at the same time, the first heating plate 120 and the second heating plate 130 start heating up. The “closed” button of the locking device 160 serves as not only a button for fixing the movement of the first heating plate 120, but also a button for starting heating.

At this time, the first heating steam button 122 and the second heating steam button 132 may be configured to control only heating operations of the first heating plate 120 and the second heating plate 130. That is, when the locking device 160 is in the “closed” state in a state in which the first heating steam button 122 is pressed and the second heating steam button 132 is not pressed, only the first heating plate 120 may start heating up. In a state in which the first heating steam button 122 is not pressed and the second heating steam button 132 is pressed, only the second heating plate 130 heats up, and when both the first heating steam button 122 and the second heating steam button 132 are pressed, both the first heating plate 120 and the second heating plate 130 may start heating up.

The operations of the power supply unit 151, the temperature controller 152, the steam controller 153, the first heating steam button 122, the second heating steam button 132, and the locking device 160 may be controlled by a controller arranged on at least one of the first heating plate 120 and the second heating plate 130. The controller is in cooperation with operation buttons of the power supply unit 151, the temperature controller 152, the steam controller 153, the first heating steam button 122, the second heating steam button 132, and the locking device 160, and when the operation buttons are pressed, the controller may control the operations of the power supply unit 151, the temperature controller 152, the steam controller 153, the first heating steam button 122, the second heating steam button 132, and the locking device 160.

In addition, a display unit configured to display states of the power supply unit 151, the temperature controller 152, the steam controller 153, the first heating steam button 122, the second heating steam button 132, and the locking device 160 may be provided. The display unit may perform a display operation such that a user can recognize on/off of the power supply unit 151, a temperature of the temperature controller 152, the amount of steam of the steam controller 153, whether or not the locking device 160 is locked, and the like.

A method of operating the above-described heating apparatus for developing a latent impact mark, according to an embodiment will now be described.

Referring to FIG. 6, first, a lower surface of the sample 110, which is treated with an amino acid reagent containing amino acids capable of reacting with a latent impact mark, is placed to come into contact with an upper surface of the second heating plate 130. At this time, the size of the second heating plate 130 may be larger than that of the sample 110 so that the sample 110 is entirely placed in the second heating plate 130.

When the sample 110 is placed on the second heating plate 130, a lower surface of the first heating plate 120 is brought into contact with an upper surface of the sample 110 by rotating the first heating plate 120 with respect to the second heating plate 130 (i.e., the first heating plate 120 lies in a closed state).

Subsequently, a power supply of the heating apparatus for developing a latent impact mark is turned on by operating the power supply unit 151, and a temperature and the amount of steam according to an amino acid reagent are set by the temperature controller 152 and the steam controller 153. In this regard, it may be determined whether only any one of the first heating plate 120 and the second heating plate 130 or both the first heating plate 120 and the second heating plate 130 is/are heated via the first heating steam button 122 and the second heating steam button 132.

After setting the temperature and the amount of steam, the locking device 160 performs a fixing operation such that the first heating plate 120 is not rotatable with respect to the second heating plate 130, and heating and steam supply begin.

The above-described heating apparatus for developing a latent impact mark, according to an embodiment has the following effects.

Generally, existing heat irons, which are sold in the market, are used, but in a case in which a sample has a large size, these general heat irons exhibit reduced developability due to imprecise heat treatment in processing, and apply wrinkles or the like to the sample. In addition, upper and lower surfaces of the sample cannot be heat-treated at the same time using an existing general heat iron, and thus to develop a latent impact mark, heat treatment should be performed on each of the upper and lower surfaces of the sample by using an existing general heat iron. However, it is time-consuming in developing a latent impact mark and it is impossible to perform precise heat treatment.

According to an embodiment, the sample 110 treated with an amino acid reagent may be placed between the first heating plate 120 and the second heating plate 130 and heated, thereby precisely and rapidly analyzing an impact mark. In particular, according to an embodiment, the first heating plate 120 and the second heating plate 130 may be made larger than the sample 110, and thus the sample 110 may be completely heat-treated at once.

In addition, the upper and lower surfaces of the sample 110 may be simultaneously heated by the first heating plate 120 and the second heating plate 130, respectively, and thus precise heat treatment is enabled, and whether the latent impact mark is developed or not may be identified without wrinkles within a short time period.

A modified embodiment of the above-described present disclosure will now be described.

Referring to FIG. 7, protrusions 171, which protrude from the upper surface of the second heating plate 130 and are capable of preventing movement of the sample 110, may be provided on the upper surface of the second heating plate 130. In a case in which the sample 110 is heated by the first heating plate 120 and the second heating plate 130, if the sample 110 is moved, precise heat treatment may not be performed.

As such, the protrusions 171 are configured to prevent the movement of the sample 110 while the sample 110 is heated, and the protrusions 171 may have various shapes according to the shape of the sample 110 placed on the second heating plate 130. In particular, when the sample 110 is a piece of clothing, as illustrated in FIG. 7, the protrusions 171 may extend along side lines of the piece of clothing. However, the protrusions 171 are not limited to the above example, and may also be provided in various forms at various positions of the upper surface of the second heating plate 130.

Protrusion grooves 172 are provided at a lower surface of the first heating plate 120, and have a shape corresponding to that of the protrusions 171 such that the protrusions 171 are inserted thereinto. When the protrusions 171 are provided on the second heating plate 130, an empty space may be formed between the first heating plate 120 and the second heating plate 130 due to the protrusions 171, and heat and steam supplied by the first heating plate 120 may not be accurately supplied to the sample 110 due to the empty space. Thus, to prevent an empty space from being formed between the first heating plate 120 and the second heating plate 130, grooves having a shape corresponding to that of the protrusions 171 are provided at the first heating plate 120 so that the protrusions 171 are inserted thereinto.

The first steam holes 121 are provided in the first heating plate 120 and the second steam holes 131 are provided in the second heating plate 130, and the first steam holes 121 may have a size different from that of the second steam holes 131. Since the same amount of steam may be supplied to the upper and lower surfaces of the sample 110, the first steam holes 121 and the second steam holes 131 may have the same size, but if necessary, there are cases in which the amounts of steam to be supplied to the upper and lower surfaces of the sample 110 should be differently set. In this case, the size of the first steam holes 121 may be different from that of the second steam holes 131, and thus the amounts of steam may be differently supplied to the upper and lower surfaces of the sample 110.

The locking device 160 may be provided at the handle 141 in a button form. When the locking device 160 is located at the handle 141 in a button form, in a case in which the locking device 160 is pressed, the first heating plate 120 may be rotatable with respect to the second heating plate 130, and in a case in which the button of the locking device 160 is not pressed, the first heating plate 120 may be fixed in a state in which the first heating plate 120 is not rotatable with respect to the second heating plate 130. That is, only when the first heating plate 120 is moved with the handle 141, the button of the locking device 160 may be pressed to move the first heating plate 120.

For this, the handle 141 may be connected to the connecting portion 140 via joints 142 (see FIG. 3). The locking device 160 may fix rotation of the first heating plate 120 by controlling rotation of the connecting portion 140. Thus, in a case in which the locking device 160 is installed at the handle 141 in a button form, the locking device 160 may be connected to the connecting portion 140 via the joints 142, and only when the button of the locking device 160 is pressed, the connecting portion 140 is rotatable.

The connecting portion 140 may have the following configuration. The first heating plate 120 is rotatably connected to the second heating plate 130 via the connecting portion 140. The connecting portion 140 may include a first connecting portion provided at an end of a side surface of the first heating plate 120 and having a hollow pillar shape, and a second connecting portion provided at an end of a side surface of the second heating plate 130 and having a rod shape allowing the second connecting portion to be inserted into the first connecting portion.

That is, the first heating plate 120 may be connected to the second heating plate 130 such that the second connecting portion is inserted into an internal space of the first connecting portion. Here, to render the first connecting portion rotatable, the pillar shape of the first connecting portion may be a cylindrical shape, and the rod shape of the second connecting portion may be a rod shape having a circular cross-section. As such, when the connecting portion 140 includes the first connecting portion and the second connecting portion, the first heating plate 120 is easily detachably assembled with the second heating plate 130.

As is apparent from the foregoing description, an impact mark may be precisely and rapidly analyzed by placing a sample treated with an amino acid reagent between a first heating plate and a second heating plate and heating the sample, and upper and lower surfaces of the sample may be heated at the same time, and thus whether the impact mark is developed or not may be identified without wrinkles and in a short time period.

It should be understood that embodiments described herein should be considered in a descriptive sense only and not for purposes of limitation. Descriptions of features or aspects within each embodiment should typically be considered as available for other similar features or aspects in other embodiments.

While one or more embodiments have been described with reference to the figures, it will be understood by those of ordinary skill in the art that various changes in form and details may be made therein without departing from the spirit and scope of the disclosure as defined by the following claims.

What is claimed is:

1. A heating apparatus for developing a latent impact mark, the heating apparatus comprising:
a sample treated with an amino acid reagent containing amino acids capable of reacting with a latent impact mark;
a first heating plate having a plate shape, having a first steam hole in a lower surface thereof, the first steam hole allowing steam to be discharged therethrough, and capable of being heated to a predetermined temperature;
a second heating plate having a plate shape, having a second steam hole in an upper surface thereof, the second stem hole allowing steam to be discharged therethrough, and capable of being heated to a predetermined temperature; and
a connecting portion configured to connect the first heating plate to the second heating plate such that the first heating plate is rotatable with respect to the second heating plate,
wherein the sample is inserted between the first heating plate and the second heating plate to be heated simultaneously by the first heating plate and the second heating plate.

2. The heating apparatus of claim 1, further comprising:
a temperature controller provided on the first heating plate or the second heating plate and capable of controlling a heating temperature of each of the first heating plate and the second heating plate; and
a steam controller provided on the first heating plate or the second heating plate and capable of controlling an amount of steam discharged via the first steam hole of the first heating plate and the second steam hole of the second heating plate.

3. The heating apparatus of claim 2, further comprising a locking device configured to perform a fixing operation such that the first heating plate is not rotatable with respect to the second heating plate.

4. The heating apparatus of claim 3, wherein, when the locking device performs a fixing operation such that the first heating plate is not rotatable with respect to the second heating plate, the first heating plate and the second heating plate start heating up.

5. The heating apparatus of claim 2, further comprising a first heating steam button capable of controlling operations of heating the first heating plate and discharging steam via the first steam hole, and a second heating steam button capable of controlling operations of heating the second heating plate and discharging steam via the second steam hole.

6. The heating apparatus of claim 1, wherein a size of each of the first heating plate and the second heating plate is larger than that of the sample.

7. The heating apparatus of claim 1, wherein a protrusion is provided on the upper surface of the second heating plate, the protrusion protruding from the upper surface of the second heating plate and capable of preventing movement of the sample, and a protrusion groove is provided in the lower surface of the first heating plate, the protrusion groove having a shape corresponding to that of the protrusion such that the protrusion is insertable into the protrusion groove.

8. The heating apparatus of claim 1, wherein the first steam hole and the second steam hole have different sizes.

9. The heating apparatus of claim 1, wherein the first heating plate and the second heating plate are covered with synthetic fiber.
